(19) United States
(12) Patent Application Publication
(10) Pub. No.: US 2004/0037860 A1
(43) Pub. Date: Feb. 26, 2004

(54) Free nicotine based cosmetic composition and uses thereof

(75) Inventor: Patrick Maillon, St. Etienne (FR)

Correspondence Address: HESLIN ROTHENBERG FARLEY & MESITI PC, 5 COLUMBIA CIRCLE, ALBANY, NY, 12203, US

(73) Assignee: COLUMBUS, Saint Etienne (FR)

(21) Appl. No.: 10/647,368

(22) Filed: Aug. 25, 2003

Related U.S. Application Data
(63) Continuation of application No. PCT/FR02/00625, filed on Feb. 19, 2002.

(30) Foreign Application Priority Data
Feb. 26, 2001 (FR) FR 01/02546

(51) Int. Cl. A61K 31/44; A61K 7/00

(52) U.S. Cl. 424/401; 514/343

(57) ABSTRACT

A topical cosmetic composition containing nicotine mixed with at least one cosmetically acceptable excipient, wherein the nicotine is free nicotine. The inventive cosmetic composition can especially be used in the treatment of cellulites.

The subject of the invention is a free nicotine-based composition. It also relates to the use of said composition for reducing or eliminating any localized excess fat of the cellulite type in particular. It finally relates to the cosmetic treatment of cellulite by the local application of said cosmetic composition.

What is commonly called cellulite is a localized gynoid lipodystrophy (called herein LGL), which presents no danger to health but which has esthetic consequences. When there is imbalance between the intake of fat and its use by the body, the adipocytes become excessively filled with lipids and compress the neighboring blood and lymphatic vessels, causing poor drainage. There is then retention of water. The thighs filled with fat and water become compartmentalized by a sclerosis consisting of components of the ground substance and more especially proteoglycans which are polymeric substances. These hardly elastic compartments constitute, at the surface of the skin, unevenness said to be in the form of “orange peel”, which is painful upon pinching, because the nerve threads are pulled by the adipose tissue.

Localized gynoid lipodystrophy (LGL) is an abnormal process of fat accumulation in the adipocytes of the thighs, and has, in addition to other causes, a hormonal dependence. Estrogens promote this accumulation; thus, at the time of puberty, pregnancy or the menopause, hormonal modifications in the body often induce the development of fatty tissues. A familial or ethnic trait also exists. When the LGL has been in existence for a long time, it can become indurated, the distended tissues becoming fibrous, and becomes more difficult to treat, see “Guidicelli Y.—Physiologie du tissu adipeux (données récentes) [Physiology of the adipose tissue (recent data)]. Cosmétologie 1995; 5: 46-49”.

LGL is an extremely frequent characteristic in women in Europe and North-America.

The conventional treatment is based on local techniques based on massages and drainages, the efficacy of which is limited, or even liposuction in difficult cases. However, liposuction is especially advantageous when the LGL is mainly adipose, and hardly organized.

Methods, based on mechanical massages, electrical stimulations (electrolipolysis) and ultrasound have also been developed.

The use of enzymes aimed at depolymerizing the proteoglycans have been proposed, in particular hyaluronidase, thiomucase and α-mucase.

Methods of stimulating lipolysis have even been proposed, the best known and the most widely used being that which consists in inhibiting phosphodiestherase in order to limit the rate of degradation of cyclic AMP. The activation of cyclic AMP is involved in the lipolytic activity of the adipocyte. Among the various phosphodiestherase inhibitors which have been recommended as slimming agents, there may be mentioned in particular xantine bases and more particularly theophyline, caffeine and threobromine.

The document U.S. Pat. No. 4,938,962 describes for example a cosmetic composition intended for the treatment of cellulite comprising the combination of a thioether with xanthine derivatives of the abovementioned type. The possible use of 2-benzylthioethylamine in salt, for example nicotinate, form as thioether is mentioned.

The document U.S. Pat. No. 4,938,962 describes, for its part, a cosmetic composition for the treatment of cellulite combining a metal salt of caffeine with vitamin E.

In the same perspective, the use of certain fat-soluble plant extracts which, according to a different mechanism, can also act as slimming agents, has been recommended. Among these plant extracts, there may be mentioned those of the climbing ivy, arnica, rosemary, marigold, sage, ginseng, St. John's wort, ruscus, meadowsweet and orthosiphon type, and mixtures of such plants.

The document DE4461308 thus describes an anticellulite composition combining a plant extract and a metabolism activator of the vitamin E type in salt, in particular nicotinate, form and with a draining active agent.

Despite the multitude of cosmetic techniques or compositions which are nowadays proposed, none is completely satisfactory. The applicant has therefore sought to develop novel solutions to the problem posed of combating cellulite. In this framework, it has discovered that free nicotine, that is to say nicotine base, exhibited, quite surprisingly, anticellulite effects or more generally was capable of reducing or eliminating any localized excess fat. In addition, the applicant observed that free nicotine improved, on the one hand, the firmness, and on the other hand, the softness of the epidermis and that in addition it delayed skin aging.

Free nicotine has been used for many years in smoking cessation, since the nicotine contained in tobacco smoke leads to a real physical dependence, the sudden withdrawal of which causes considerable psychological and physical disorders. Consequently, free nicotine is incorporated into topical pharmaceutical compositions of the patch or oral type, or of the chewing gum or oral pastille type. Free nicotine can in addition be administered by the transmucous route, by means for example of a nasal spray.

Regardless of the type of medicaments used, the objective of smoking cessation is to deliver a sufficient quantity of free nicotine at the cerebral level and as a whole at the systemic level so that said cessation can be effective while being controlled. That is why it is imperative to deliver a relatively large quantity of nicotine continuously or at regular intervals, from 14 to 21 mg/24 hours or even more in some cases of high nicotine dependence. The doses are then reduced so as to make the cessation permanent.

Although some prior art documents, in particular the documents U.S. Pat. No. 4,938,962 and DE-440308, describe the use of nicotine for the treatment of cellulite, this systematically involves nicotine derivatives used in salt, in particular nicotinate, form. Also, nothing in these documents would suggest to a person skilled in the art to use, instead of salts, free nicotine, that is to say nicotine base.

It was not therefore evident, in the light of the state of the art known to a person skilled in the art, that free nicotine can be used in the cosmetic field, in particular for the treatment of cellulite.

The subject of the invention is therefore a topical cosmetic composition comprising nicotine in the form of a mixture with at least one cosmetically acceptable vehicle, which is characterized in that the nicotine is free nicotine.

According to a first characteristic of the invention, the free nicotine represents between 0.001 and 0.5% by weight of the composition.

For a value of less than 0.001, no convincing effect is observed. For a value greater than 0.5%, there is an exposure to the risk of substantial systemic passage of free nicotine.

The topical composition of the invention is used as a local application and may consequently be provided in various forms such as a gel, oily gel, cream, ointment, milk, lotion, balm, spray, emulsion, patch, stick.

When the composition is an emulsion, the proportion of the fatty phase may range from 5% to 80% by weight, and preferably from 5% to 50% by weight relative to the total weight of the composition. The oils, waxes, emulsifiers and coemulsifiers used in the composition in the form of an emulsion are chosen from those conventionally used in the cosmetic field. The emulsifier and the coemulsifier are present in the composition in a proportion ranging from 0.3% to 30% by weight, and preferably from 0.5 to 20% by weight relative to the total weight of the composition.

In a known manner, the cosmetic composition may also contain adjuvants which are customarily used in the cosmetic field, such as hydrophilic or lipophilic gelling agents, hydrophilic or lipophilic additives, preservatives, antioxidants, solvents, perfumes, fillers, screening agents, odor absorbers and coloring matter. The quantities of these various adjuvants are those conventionally used in the cosmetic field, and for example from 0.01% to 10% of the total weight of the composition.

As oils or waxes which can be used in the invention, there may be mentioned mineral oils, vegetable oils, animal oils, synthetic oils, silicone oils or waxes and beeswax, carnauba wax or paraffin wax. It is possible to add fatty alcohols and fatty acids (stearic acid) to these oils.

As solvents which can be used in the invention, there may be mentioned low alcohols, in particular ethanol and isopropanol, and propylene glycol.

As hydrophilic gelling agents which can be used in the invention, there may be mentioned carboxyvinyl polymers (carbomer), acrylic copolymers such as acrylate/alkyl acrylate copolymers, polyacrylamides, polysaccharides such as hydroxypropylcellulose, natural gums and clays, and as lipophilic gelling agents, there may be mentioned modified clays such as bentones, metal salts of fatty acids such as aluminum stearates and hydrophobic silica, ethyl cellulose, polyethylene.

As already stated, the applicant has discovered that free nicotine acts on cellulite, and the firmness, softness and aging of the skin.

Consequently, the invention also relates to the use of the composition as described above, for reducing or limiting any localized excess fat of the cellulite type in particular.

It is quite obvious that in no case can this use be considered as corresponding to a therapeutic treatment since the local application of free nicotine is not intended to avoid a pathological state but on the contrary to improve a condition which is purely of an esthetic nature.

The use of free nicotine is also envisaged for the manufacture of a composition intended for reducing or limiting any localized excess fat of the cellulite type in particular.

The invention also relates to the use of the cosmetic composition described earlier for increasing the firmness of the epidermis. This property is particularly advantageous for the treatment of wrinkles.

Likewise, it relates to the use of the composition described above for combating skin aging.

According to another aspect, it relates to the use of the composition described above for improving the softness of the epidermis.

The invention also relates to a method for the cosmetic treatment of local excess fat of the cellulite type in particular, according to which an effective quantity of the composition described earlier is applied locally, by the topical route.

In practice, the quantity of free nicotine does not exceed 5 mg per application; it is advantageously between 0.3 and 3 mg.

The invention and the advantages attached thereto will emerge clearly from the following exemplary embodiments.

### EXAMPLE 1

### Comparative Trial

1/ Introduction

The applicant carried out a comparative trial intended to evaluate the efficacy of the composition of the invention, as a local topical application, in patients with gynoid lipodystrophy of the thighs and of the buttocks, in comparison with a placebo, considered as a control group. Concomitantly, the effects of the composition on the softness and firmness of the skin were assessed.

The dose of nicotine delivered through the skin is 0.5 mg per side (top of the thigh and buttocks), compared with the LGL zones, at the rate of two applications per day, for 56 days.

The formulas of the compositions applied are the following:

| | | COMPOSITION OF |
|---|---|---|
| | PLACEBO (g) | THE INVENTION (g) |
| Ethyl alcohol 95% | 50 | 50 |
| Isopropyl alcohol | 5 | 5 |
| Carbopol 980 | 1 | 0.5 |
| Glycerol | 10 | 10 |
| Propylene glycol | 10 | 10 |
| Trolamine | 0 | 0.2917 |
| Nicotine base | 0 | 0.0383 |
| (Mw = 162.23 g) | | |
| Water | 24 | 24.17 |
| Total | 100 | 100 |

2/ Method

2.1/ Objectives of the Study

The objectives of this clinical trial, carried out on patients suffering from gynoid lipodystrophy of the thighs and buttocks, are the following:

Main objective: To compare the efficacy of the composition of the invention to a topical placebo,

Secondary objective: To evaluate the variation of the LGL tolerance, as well as the satisfaction, the local, general, clinical and biological tolerance of the product, and in particular its safety. To evaluate the softness and the suppleness of the skin, its firmness, the reduction of the number and of the depth of the nodes of fat.

2.2/ Study Profile

It is a randomized comparative double blind monocenter trial comprising two groups of treatment of localized gynoid lipodystrophy of the thighs and of the buttocks (LGL):

Group 1: Treatment with the composition of the invention (verum)

Group 2: Treatment with a continuous topical placebo

This trial lasted for 9 weeks and comprised a one-week inclusion phase, followed by an 8-week treatment phase. It was carried out in patients suffering from LGL.

The total number of patients in this preliminary study is 40.

2.3/ Efficacy Criteria

Evaluation carried out during the inclusion visit, at D28, and at the final visit for the principal and secondary criteria.

2.3.1 Principal Criterion

1.-Measurement of the adipose panicle by ultrasound scan, measured with the aid of a wide probe, by looking at the great trochanter of each femur. “Diridollou S. Gall Y.—Exploration ultrasonore de la peau: des débuts prometteurs [Ultrasound exploration of the skin: promising beginnings]. Cosmétologie 1998; 17: 40-45”, “Ohnuma M., Ashizawa K.—A—Mode Type Ultrasonique and Caliper Measurments of Subcutaneous Adipese Tissue Thickness. J. Human Zrgol., 1988; 17 : 97-100”, “Schnebert S., Perin F., Pittet J.-C., Beau P., Pourcelot Léandre—Echographie, une technique accessible et fiable pour mesurer l'efficacité des produits amincissants [Ultrasound scan, an accessible and reliable technique for measuring the efficacy of slimming products]. Cosmétologie 1999; 22: 35-38”.

2. The probe was placed on a tripod, at the constant level of the ground for each subject. The measurement was carried out after acquiring the last possible image by detaching the probe from the skin plane.

2.3.2 Secondary Criteria

Subjective assessment of the patient on the variation of her LGL, evaluated by visual analogic scales.

Variation of the subjective tolerance of the patient to her LGL, evaluated by visual analogic scales.

Subjective assessment of the patient on the variation of the firmness of her skin, the number and depth of the nodes of fat, by a visual analogic scale.

Assessment of the suppleness and softness of the skin by a daily monitoring sheet.

2.4/ Acceptability

Average overall satisfaction of the treatment of the LGL in the patients of groups 1 and 2, evaluated during the final visit and measured with a visual analogic scale coding the satisfaction from 1 to 15.

Disruption caused by the treatment in the daily activity, evaluated during the final visit, and described as mild (not affecting daily activity), moderate (not greatly affecting daily activity, but requiring more rest during the day), severe (affecting daily activity, by requiring prolonged bed rest).

2.5/ General Tolerance

The general tolerance was evaluated during the final visit. A biological assessment was carried out during the selection visit and the final visit (blood count, platelet count, transaminases ALAT and ASAT).

2.6/ Satisfaction

The satisfaction of the patients in both groups was evaluated through a question on the rate of recommendation of the product by the visual analogic scale.

3/ Inclusion and Noninclusion Criteria

3.1/ Inclusion Criteria

women aged at least 18,

patient suffering from LGL,

patient wishing to see a reduction in their LGL, and accepting to comply with the treatment and protocol conditions,

patient whose body mass index is less than 30 (BMI=W/H2),

patient not regularly consuming, or who has never regularly consumed, tobacco in any form whatsoever (cigarette, cigar, pipe, chewing tobacco, snuff, and the like),

patient with no cardiac, pulmonary, gastrointestinal, hepatic, renal, hematological or neurological condition determined by anamnesis or by clinical examination,

patient likely not to be pregnant during the trial.

3.2/ Noninclusion Criteria

patient having a history of hypersensitivity to nicotine and to other constituents of the product studied,

patient using or who has used a nicotine product (patch, chewing gum, spray and the like) in the last two years,

patient having or having had a morbid obesity with a body mass index greater than 30,

patient who has had a serious condition requiring continuous treatment or supervision,

patient having or having had a skin condition with or without a systemic consequence,

participation in another clinical trial within the three months preceding the present trial,

patient whose biological examination for selection is not within the limits of the standards set, blood count, SGOT, SGPT),

patient who has had a recent surgical operation,

patient who, in the opinion of the experimenter, would not be capable of following the instructions for the protocol.

4/ Results

All the results presented here include the entire population which followed the complete protocol.

Number of patients: 40 randomized patients in a single center, 20 received a treatment with nicotine gel and 20 a placebo gel.

4.1/ Demographic Criteria

They are similar in both groups of 20:

TABLE 1
Demographic criteria and inclusion characteristics

| | | Verum | | Plac bo | | Total | |
|---|---|---|---|---|---|---|---|
| Age | Mean | 40.683 | (8.955) | 41.408 | (9.965) | 41.046 | (9.358) |
| | (standard | | | | | | |
| | deviation) | | | | | | |
| | Minimum | 21.3 | | 18.3 | | 18.3 | |
| | Maximum | 53.2 | | 52.8 | | 53.2 | |
| Weight | Mean | 65.950 | (6.646) | 66.235 | (9.659) | 99.092 | (8.133) |
| | (standard | | | | | | |
| | deviation) | | | | | | |
| | Minimum | 57 | | 48.8 | | 48.8 | |
| | Maximum | 80.5 | | 85.5 | | 85.5 | |
| BMI | Mean | 25.186 | (2.301) | 25.331 | (3.217) | 25.259 | (2.762) |
| | (standard | | | | | | |
| | deviation) | | | | | | |
| | Minimum | 21.5 | | 19.5 | | 19.5 | |
| | Maximum | 29 | | 29.9 | | 29.9 | |
| Thickness of LGL | Mean | 90.44 | (16.62) | 86.61 | (30.32) | 88.53 | (24.21) |
| measured on both | (standard | | | | | | |
| thighs | deviation) | | | | | | |
| | Minimum | 63.4 | | 33.0 | | 33 | |
| | Maximum | 124.2 | | 142.8 | | 142.8 | |
| Perimeter of both | Mean | 99.57 | (6.99) | 99.962 | (7.62) | 99.60 | (7.22) |
| thighs (bottom) | (standard | | | | | | |
| | deviation) | | | | | | |
| | Minimum | 87.5 | | 85.5 | | 85.5 | |
| | Maximum | 118.0 | | 114.5 | | 118.0 | |
| Perimeter of both | Mean | 118.88 | (7.70) | 117.50 | (9.16) | 118.19 | (8.38) |
| thighs (top) | (standard | | | | | | |
| | deviation) | | | | | | |
| | Minimum | 107.5 | | 99.5 | | 99.5 | |
| | Maximum | 137 | | 139 | | 139 | |
| Bitrochanteric | Mean | 102.00 | (4.87) | 102.53 | (7.05) | 102.26 | (5.99) |
| perimeter | (standard | | | | | | |
| | deviation) | | | | | | |
| | Minimum | 94.0 | | 90.0 | | 90.0 | |
| | Maximum | 112.5 | | 115.5 | | 115.5 | |

BMI = body mass index (W/H2)

4.2/ Principal Criteria

The criterion for efficacy selected is the 5% reduction of the LGL between D0 and D56. A chi-square test was carried out; it shows a significant difference at p=0.006.

TABLE 2
Loss of thickness between the treated group and the placebo group

| | |
|---|---|
| Verum | Placebo |

Variation at D28 of the ultrasound scan thickness of the LGL in %

| | | |
|---|---|---|
| Mean (standard deviation) | −5.54% (5.06) | +0.61% (2.09) |
| Minimum | −0.19% | +3.84% |
| Maximum | −20.92 | −4.66% |

Variation at D56 of the ultrasound scan thickness of the LGL in %

| | | |
|---|---|---|
| Mean (standard deviation) | −8.90% (9.05) | −0.85% (4.33) |
| Minimum | −0.15% | +5.39% |
| Maximum | −39.03% | −13.10% |

These mean values are significantly different between the verum and the placebo.

4.3/ Secondary Criteria

There is a significant difference (p≦0.05) between the two groups as regards the assessment of the variation of the cellulite, on the efficacy of the product, the firmness of the skin, the reduction in the number and in the depth of the nodes of fat, and the sensation on wearing tailored clothes.

TABLE 3
Questionnaire by visual analogic scale at D28

| | |
|---|---|
| Verum | Placebo |

Did you find the gel which you have used effective?

| | | |
|---|---|---|
| Mean (standard deviation) | 7.20 (2.57) | 4.82 (3.32) |
| Minimum | 1.3 | 0.0 |
| Maximum | 11.2 | 11.9 |

TABLE 4
Questionnaires by the visual analogic scale at D56

| | |
|---|---|
| Verum | Placebo |

Did you find the gel which you have used effective overall?

| | | |
|---|---|---|
| Mean (standard deviation) | 9.17 (3.34) | 5 (3.99) |
| Minimum | 2.7 | 0.6 |
| Maximum | 14.1 | 13.9 |

Did you find it effective for reducing the nodes of fat (number and depth)?

| | | |
|---|---|---|
| Mean (standard deviation) | 8.68 (3.52) | 5.14 (4.28) |
| Minimum | 1.9 | 0.0 |
| Maximum | 14.2 | 14.3 |

Did you find it effective on the firmness of your skin?

| | | |
|---|---|---|
| Mean (standard deviation) | 9.93 (3.05) | 6.74 (4.81) |
| Minimum | 3.6 | 0.4 |
| Maximum | 14.1 | 14.9 |

Did you have the impression of feeling better in your tight-fitting clothes?

| | | |
|---|---|---|
| Mean (standard deviation) | 9.76 (3.74) | 5.80 (5.13) |
| Minimum | 2.8 | 0.0 |
| Maximum | 14.9 | 14.8 |

These subjective analogic questionnaires are in agreement with the measurements carried out.

The daily sheets show an improvement in the softness of the skin, and its suppleness.

4.4/ Tolerance

Reports of itching or prickling at the site of application are negligible. The rare reports are quoted ⅓, and of short duration; they were spontaneously resolvent and did not cause termination of the treatment to be envisaged. No redness is reported.

The vital parameters are not significantly modified by the nicotine gel.

4.5/ Satisfaction

The satisfaction of the patients was evaluated by an analogic scale.

There is a significant difference in the responses between the two groups, both at D28 and at D56.

TABLE 5
“Would you recommend this gel?”

| | | Verum | Placebo |
|---|---|---|---|
| D28 | Mean (standard deviation) | 9.60 (3.62) | 7.09 (4.13) |
| | Minimum | 1.0 | 0.0 |
| | Maximum | 14.9 | 15 |
| D56 | Mean (standard deviation) | 12.11 (2.38) | 7.11 (5.27) |
| | Minimum | 7.9 | 1.3 |
| | Maximum | 15.0 | 14.9 |

There is an increase in the mean of the satisfaction index and an increase in the significance between the two groups between D28 (p=0.044) and D56 (p=0.006).

5/ Discussion

Nicotine, used in a small dose, acts directly on localized gynoid lipodystrophy, more commonly called cellulite, by significantly reducing its thickness (by 8.9% on average). The entire population studied lost weight (−1.37 kg in the verum group, against −0.71 kg in the placebo group). There is no significant difference between these two groups for this criterion, either at D28 or at D56, or when noting the variation. The free nicotine used in the context of this study therefore shows no overall slimming effect. It can therefore be concluded therefrom that the effect of nicotine is localized and nonsystemic. The variation in the thickness of cellulite cannot therefore be attributed to the slimming noted in the two groups.

The patients noted that the nicotine gel was effective overall in reducing the extent of their cellulite, in reducing the number and the depth of the nodes of fat. This variation is noticeable when the wearing of tailored clothes.

The effect of the nicotine gel on the firmness, tone and softness of the skin was finally noted.

The tolerance and the satisfaction of the patients is good.

The agreement between the results measured, the evaluations of the patients by the visual analogic scale and the satisfaction index should be noted.

### EXAMPLE 2

### Formulation

| | g % g |
|---|---|
| Slimming gel | |
| Ethyl alcohol 95% | 45 |
| Isopropyl alcohol | 5 |
| Carbopol 980 | 2 |
| Glycerol | 10 |
| Propylene glycol | 10 |
| Trolamine | 0.3 |
| Nicotine base (MW = 162.23 g) | 0.03 |
| Water | qs 100.00 |
| Sliming balm | |
| Natural ozokerite | 15.00 |
| Liquid purcellin oil | 10 |
| Petroleum jelly | 15 |
| Trolamine | 0.5 |
| Nicotine base (MW = 162.23 g) | 0.03 |
| Preservative | QS |
| Antioxidant | QS |
| Liquid paraffin | qs 100.000 |
| Thermoslimming body oil | |
| Hexyl nicotinate | 3 |
| “Miglyol 812 (fatty acid | 15 |
| triglycerides) from the | |
| company DYNAMIT NOBEL | |
| Isopropyl palmitate | 10 |
| Sweet almond oil | 8 |
| Trolamine | 0.5 |
| Nicotine base (MW = 162.23 g) | 0.03 |
| Preservative | QS |
| Perfume | QS |
| Antioxidant | QS |
| Petroleum jelly | 100.000 |
| Toning mousse | |
| Cationic polymer “CELQUAT L-200” | 2 |
| from the company NATIONAL STARCH | |
| Surfactant (TWEEN 20) | 3 |
| Oxyethylenated nonyphenol | 10 |
| (12 mols of ethylene oxide) | |
| Trolamine | 0.5 |
| Nicotine base (MW = 162.23 g) | 0.03 |
| Glycerin | 5 |
| Preservative | QS |
| Ethyl alcohol | qs 100.00 |

This composition is intended to be packaged as an aerosol in the presence of a propelling agent, butane for example.

Antiwrinkle cream

| | g % g |
|---|---|
| Glyceryl stearate | 0.5 |
| Polysorbate 60 (“Tween 60” | 0.5 |
| sold by the company ICI | |
| Stearic acid | 4 |
| Triethanolainine | 3 |
| Resveratrol | 3 |
| Nicotine base (MW = 162.23 g) | 0.03 |
| Carbomer | 4 |
| Shea butter | 15 |
| Perhydrosqualene | 15 |
| Perfume | QS |
| Preservative | QS |
| Water | qs 100.000 |

Oil-in-water emulsion prepared in a conventional manner for persons skilled in the art.

Anti-orange peel skin cream

| | g % g |
|---|---|
| Trolamine | 0.5 |
| Nicotine base (MW = 162.23 g) | 0.03 |
| Sodium dimethicone copolyol | 5 |
| acetal methyl taurate | |
| (Pecosil DCT from the | |
| company PHOENIX) | |
| Mineral oil | 15 |
| Glycerin | 5 |
| Perfume | QS |
| Preservative | QS |
| Water | qs 100.000 |

Oil-in-water emulsion prepared in a conventional manner for persons skilled in the art.

1/ A topical cosmetic composition comprising nicotine in the form of a mixture with at least one cosmetically acceptable vehicle, characterized in that the nicotine is free nicotine.

2/ The composition as claimed in claim 1, characterized in that the free nicotine represents between 0.001 and 0.5% by weight of the composition.

3/ The composition as claimed in either of the preceding claims, characterized in that it is in the form of a gel, oily gel, cream, ointment, milk, lotion, balm, spray, emulsion, patch, stick.

4/ The use of the composition as claimed in one of the preceding claims, for reducing or limiting any localized excess fat of the cellulite type in particular.

5/ The use of the composition as claimed in one of claims 1 to 3, for improving the firmness of the epidermis.

6/ The use of the composition as claimed in one of claims 1 to 3, for combating skin aging.

7/ The use of the composition as claimed in one of claims 1 to 3, for improving the softness of the epidermis.

8/ A method for the cosmetic treatment of local excess fat of the cellulite type in particular, according to which an effective quantity of the composition which is the subject of one of claims 1 to 3 is applied locally, by the topical route.

9/ The method as claimed in claim 8, characterized in that the quantity of nicotine released is between 0.3 and 3 mg per application.
